(19) United States
(12) Patent Application Publication
(10) Pub. No.: US 2012/0041490 A1
(43) Pub. Date: Feb. 16, 2012

(54) VARIABLE OFFSET SPINE FIXATION SYSTEM AND METHOD

(75) Inventors: R. Patrick Jacob, Gainesville, FL (US); Eric McDivitt, West Chester, PA (US); Joseph Capozzoli, West Chester, PA (US); Thomas Keyer, West Chester, PA (US)

(73) Assignee: Synthes USA, LLC, West Chester, PA (US)

(21) Appl. No.: 13/002,602

(22) PCT Filed: Nov. 18, 2009
(86) PCT No.: PCT/US2009/006176

§ 371 Date: Oct. 18, 2011

(51) Int. Cl. A61B 17/70; A61B 17/88; A61B 17/86

(52) U.S. Cl. 606/264; 606/305; 606/279

(57) ABSTRACT

A minimally invasive system and method for coupling a spinal rod to a plurality of bone anchors implanted into a plurality of vertebral bodies. A plurality of bottom-loading polyaxial anchor seat assemblies having different vertical heights are chosen to pop over the heads of the implanted bone anchors and a spinal rod is more easily introduced and secured to the bone anchors. The variety of different heights that characterize the plurality of polyaxial anchor seat assemblies allows a surgeon to intraoperatively choose the appropriate offset for a particular spinal level during spinal corrections.

### BACKGROUND OF THE INVENTION

Minimization of surgical steps during posterior screw and rod placement during some spinal surgeries is desirable. Current top loading polyaxial pedicle screw systems, in which the bone anchor is loaded manually into the down through the top of the anchor seat just prior to the surgery or, more often, in which the combination comes preassembled, typically requires several surgical steps after an incision and access path are provided. These steps may include implantation of multiple polyaxial screw and anchor seat assemblies, insertion of a spinal rod into the multiple screw and anchor seat assemblies insertion of a locking cap into each screw and anchor seat assembly and tightening of the locking cap onto each screw and anchor seat assembly for locking the construct. Seating of the rod and insertion of the multiple locking caps can be challenging due to the anatomical placement of the bone anchors, the nature of the anatomical correction required, less than optimal rod bending, and other factors that can complicate the reduction of the rod into the anchor seats. Complicated rod reduction may result incross-threading of the locking screw, anchor seat damage and potential replacement of the pedicle screw and anchor seat assembly mid-surgery. Preassembled screw and anchor seat assemblies have a profile that reduces the surgeon's visibility during its implantation and, further, its footprint, once implanted, decreases the amount of working space available in the anatomy. Surgeons often struggle to achieve full rod reduction or anatomical correction even using bottom-loading, pop-on head type pedicle screw systems. While powerful rod reduction instruments are available for use with the rod and screw construct systems, the surgeon is often not able to completely reduce the rod into the anchor seat without pre-bending and re-bending the rod to the point at which the rod may become weakened. Further, the application of too much force during the reduction of the spinal rod may also cause one or more of the implanted bone anchors to be dislodged from the vertebral bodies in which they are implanted. It is thus desirable to develop a system and associated method that provides for a reduction in the difficulty and number of surgical steps necessary during posterior screw and rod placement during spine surgery when patient anatomy results in a complicated construct.

### BRIEF SUMMARY OF THE INVENTION

The present invention relates generally to orthopedics. More specifically, the present invention relates to a posterior spine fixation system and method including an assembly of variable height anchor seats.

In a preferred embodiment of the present invention, a kit includes a plurality of bone anchors, each bone anchor having a polyaxial head and a shaft, and a plurality of anchor seat assemblies. Each anchor seat assembly includes a proximal end and a distal end, and each of the plurality of anchor seat assemblies further includes a rod receiving channel that has a channel proximal end adjacent the proximal end of the anchor seat assembly and a channel distal end terminating between the anchor seat proximal end and the anchor seat distal end. At least two of the plurality of anchor seat assemblies have different heights between the distal end of the anchor seat assembly and the channel distal end of the rod receiving channel. The kit further includes a plurality of locking caps couplable to the plurality of anchor seat assemblies adjacent the anchor seat proximal end adapted to secure a spinal rod with respect to the plurality of bone anchors within one of the rod receiving channels of the plurality of anchor seat assemblies.

A method includes anchoring a plurality of bone anchors into the plurality of vertebral bodies and selecting a plurality of anchor seat assemblies, where at least two of the plurality of anchor seat assemblies have different heights. The method further includes coupling the plurality of anchor seat assemblies to the plurality of bone anchors, coupling a spinal rod to the plurality of anchor seat assemblies, and locking the anchor seat assemblies to the spinal rod with a plurality of locking caps.

### BRIEF DESCRIPTION OF THE SEVERAL VIEWS OF THE DRAWINGS

The foregoing summary, as well as the following detailed description of the preferred embodiments of the application, will be better understood when read in conjunction with the appended drawings. For the purposes of illustrating the kit of the present application, there is shown in the drawings preferred embodiments. It should be understood, however, that the application is not limited to the precise arrangements and instrumentalities shown. In the drawings:

FIG. 1 illustrates front elevational views of several pedicle screw assemblies of a spine fixation system, in accordance with one or more embodiments of the present application;

FIG. 2 illustrates a side elevational view of the spine fixation system including pedicle screw assemblies in accordance with FIG. 1;

FIG. 3 illustrates an exploded perspective view of a spine fixation system in accordance with one or more embodiments of the present application;

FIG. 4 illustrates a cross-sectional view of a portion of a spine fixation system in accordance with one or more embodiments of the present application;

FIG. 5 illustrates a cross-section view of an anchor seat in accordance with one or more embodiments of the present application;

FIG. 6 illustrates a cross-section view of an anchor seat in accordance with one or more embodiments of the present application;

FIG. 7 illustrates a cross-section view of an anchor seat in accordance with one or more embodiments of the present application; and

FIG. 8 illustrates a perspective view of a spine fixation system in accordance with one or more embodiments of the present application.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” or “distally” and “outwardly” or “proximally” refer to directions toward and away from, respectively, the geometric center of the spine fixation system and related parts thereof. The words, “anterior”, “posterior”, “superior,” “inferior” and related words and/or phrases designate preferred positions and orientations in the human body to which reference is made and are not meant to be limiting. The terminology includes the above-listed words, derivatives thereof and words of similar import.

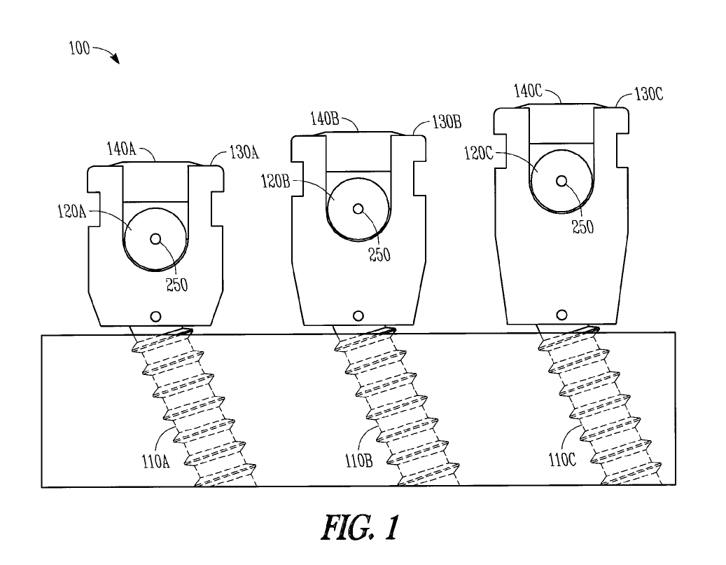

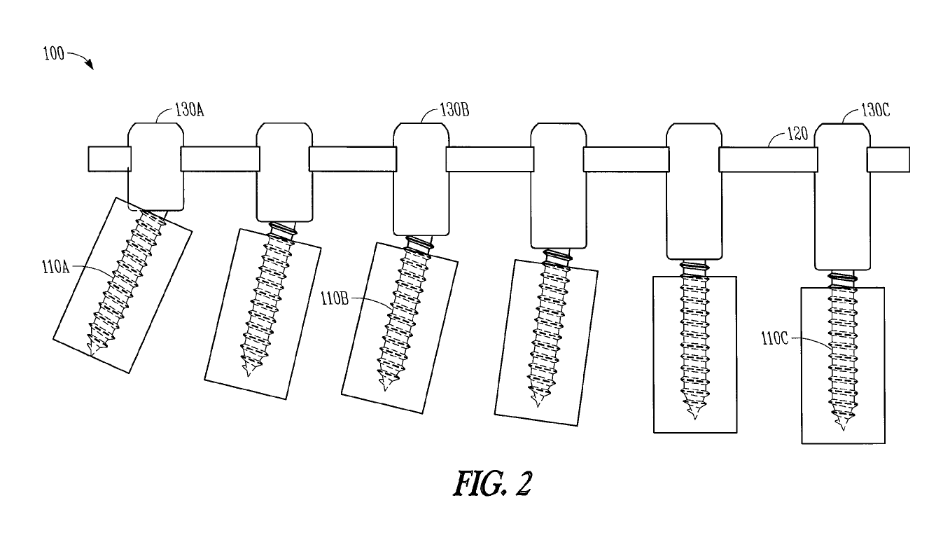

Referring to FIGS. 1 and 2, a spine fixation system 100 is provided that includes a plurality of bone anchors 110A, 110B, 110C, a plurality of anchor seats 130A, 130B, 130C, a plurality of collets 152, and a plurality of locking caps 140A, 140B, 140C. The system 100 also preferably includes at least one spinal rod 120. The bone anchor 110A, 110B, 110C, anchor seat 130A, 130B, 130C, locking cap 140A, 140B, 140C and collet 152 are generally considered to make-up a pedicle screw construct.

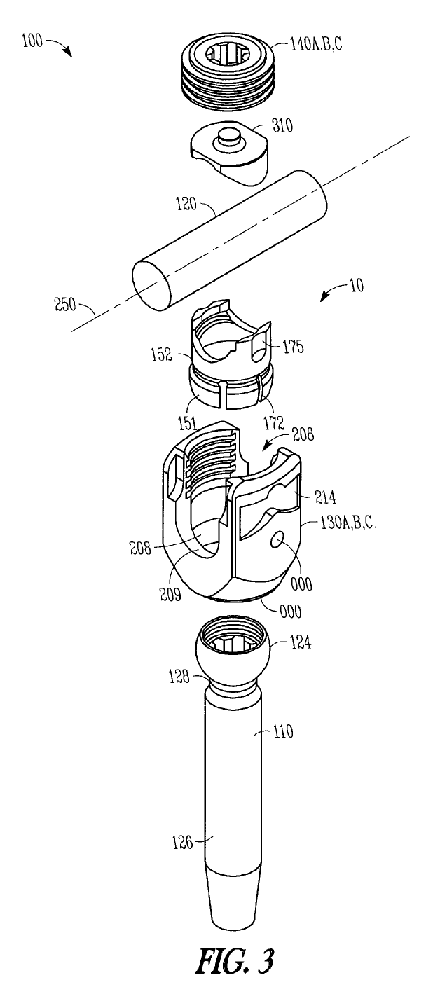

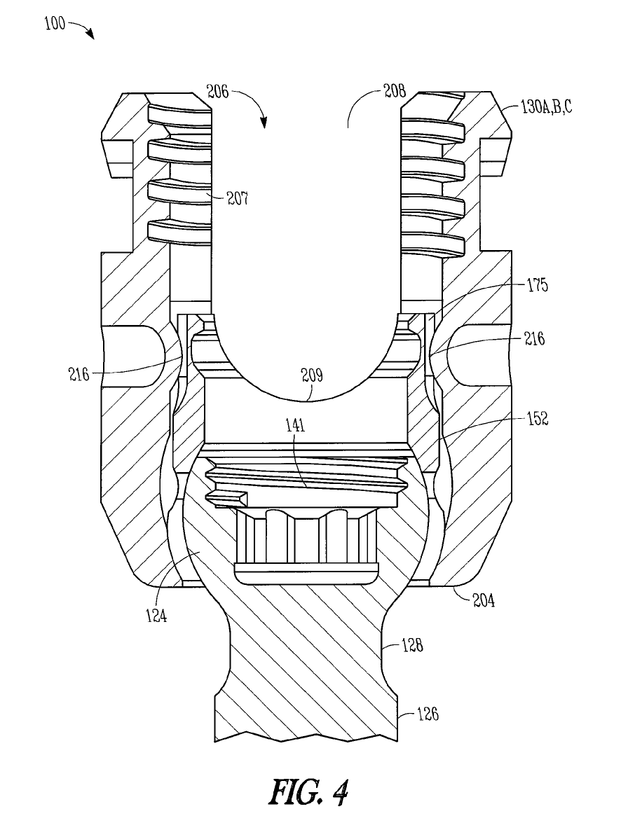

Each of the plurality of bone anchors 110A, 110B, 110C includes a threaded shaft 126 and a partially spherical or otherwise enlarged heads 124, as shown in FIG. 3. The frustaspherical head 124 assists in facilitating rotation with respect to the collet 152. The head portion 124 includes a driver portion that receives a portion from a driver for rotating the bone anchor 110 into engagement with a patient's vertebra V (FIGS. 2 and 8). The bone anchor 110 may include a reduced diameter neck portion 128 between the head 124 and the shaft 126. One or more of the bone anchors 110A, 110B, 110C may include an interior cannulation to accommodate implantation of the bone anchor 110A, 110B, 110C over a guide wire.

Each of the plurality of anchor seats 130A, 130B, 130C includes an axial bore 206 and a rod receiving channel 208 oriented transversely with respect to the axial bore 206. The anchor seat 130A, 130B, 130C preferably includes interior threading 207 for receiving the externally threaded locking cap 140A, 140B, 140C, but may alternatively include external threading or a locking feature (not shown) to engage the locking cap 140A, 140B, 140C.

The distal portion of the axial bore 206 is configured to house the collet 152. The collet 152 optionally includes a partially spherical interior volume at a distal end for capturing and retaining the head portion 124 of the bone anchor 110A, 110B, 110C and a plurality of deflectable fingers that are positioned at the distal end. The collet 152 is preferably configured to pop over the head of the bone anchor 110A, 110B, 110C via the deflection of the fingers and to retain the head of the bone anchor 110A, 110B, 110C therein, while permitting articulation and rotation of the anchor seat 130A, 130B, 130C, collet 152 and locking cap 140A, 140B, 140C with respect to the bone anchor 110A, 110B, 110C.

The fingers 172 of the collet 152 are configured to collapse around the head 124 of the bone anchor 110A, 110B, 110C and lock the articulation and rotation of the anchor seat 130A, 130B, 130C with respect to the bone anchor 110A, 110B, 110C as a result of a downward force applied to the top of the collet 152 that causes the exterior surface of the fingers to interact with the interior surface of the distal portion of the anchor seat portion 130A, 130B, 130C and thereby crush lock the collet 152 around the head of the bone anchor 110A, 110B, 110C. An outer surface of the fingers 172 includes a spherical convex surface 151 of the collet 152 that contacts an inner surface of the anchor seats 130A, B, C.

The preferred collet 152 includes one or more grooves 175 on an outer surface that engage a projection or dimple 216 of the anchor seat 130A, B, C. The collet 152 optionally floats within the axial bore 206 of the anchor seat between a loading position and a locked position. Interaction between the one or more grooves 175 and the projection or dimple 216 assists in preventing the collet 152 from moving out of the upper end of the anchor seat when in the loading position. The collet 152 optionally further includes one or more rod-locking features such that the spinal rod 120 is coupled to the collet 152, and relative to the anchor seat.

The locking cap 140A, 140B, 140C is preferably externally threaded, includes an instrument engagement feature and may further include an axial bore. The locking cap 140A, 140B, 140C may further include a rotatably coupled saddle element 310 configured to contact the top surface of the rod 120 and freely rotate with respect to the locking cap 140A, 140B, 140C. The saddle 310 further includes a recess that receives at least a portion of the rod 120. The locking cap 140A, 140B, 140C is configured to be engaged and rotated with respect to the anchor seat 130A, 130B, 130C using a driver instrument. As the locking cap 140A, 140B, 140C is driven down into the anchor seat 130A, 130B, 130C, the bottom surface of the locking cap 140A, 140B, 140C bears against the top of the rod 120 and applies a downward force to the top of the collet 152 to lock the angulation of the anchor seat 130A, 130B, 130C with respect to the bone anchor 110A, 110B, 110C. An intermediary wedge member may be included between the collet 152 and the rod 120 or between the collet 152 and the locking cap 140A, 140B, 140C to assist in directing the downward force from the advancement of the locking cap 140A, 140B, 140C to the top of the collet 152. Alternatively, the collet 152 may include arm members or other features that extend upwardly and interact directly with the bottom of the locking cap 160.

As shown in FIGS. 1 and 2, the spine fixation system 100 includes variable height pedicle screw assemblies wherein multiple anchor seats 130A, 130B, 130C are provided that have different heights between the bottom 209 of their rod receiving channels and the distal end 204 of each anchor seat 130A, 130B, 130C when the rod 120 is in a seated position. Similarly, the corresponding collets 152 positioned in the anchor seats 130A, 130B, 130C between the rod-contacting surfaces of the collets 152 and the collapsible bone anchor head-grasping portion of each collet 152 have different heights. Alternately, in the case in which an intermediary wedge member is disposed within each anchor seat 130A, 130B, 130C between the collet 152 and the rod 120, the lengths of the collets 152 and/or the intermediary wedge members can vary between each anchor seat 130A, 130B, 130C. FIG. 1 illustrates three anchor seats 130A, 130B, 130C having heights from near a center of the bone anchor 110A, 110B, 110C to a longitudinal axis 250 of the spinal rod 120. In an assembled configuration, the heights may be comprised of five, nine and thirteen millimeters (5 mm, 9 mm and 13 mm), as can be seen in FIG. 1. The pedicle screw assemblies are not limited to these heights and the kits of the spinal fixation system 100 of the present application are not limited to having three heights and the kits may include pedicle screw assemblies having nearly any number of heights and dimensions that are adaptable to the anatomy encountered in patients who receive spinal surgery such that the rod receiving channels of the constructs may be linearly aligned in a final construct, as will be described in greater detail below. The constructs can be used for a curved rod and/or the kits can be used for sagittal correction or coronal correction.

The pedicle screw assemblies are preferably provided to a surgeon in a kit or tray including a plurality of bone anchors 110A, 110B, 110C having different lengths and/or diameters, a plurality of anchor seats 130A, 130B, 130C having various heights with collets 152 therein, a plurality of locking caps 140A, 140B, 140C, which are, in an option, mountable to any of the anchor seats 130A, 130B, 130C and a plurality of rods 120 having various lengths and/or diameters. Following insertion of the bone anchors 110A, 110B, 110C into the vertebrae V the surgeon selects the appropriate anchor seats 130A, 130B, 130C to mount to the bone anchors 110A, 110B, 110C, selecting the appropriate height anchor seat 130A, 130B, 130C that permits alignment of the rod receiving channels of the anchor seats 130A, 130B, 130C, which minimizes bending of the spinal rod 120. That is, the heights of the anchor seats 130A, 130B, 130C are selected such that the rod receiving channels of the anchors seats 130A, 130B, 130C are generally linearly aligned such that bending of the spinal rod 120 is minimized. Similarly, in cases where the spinal rod 120 is further from the bone anchors 110A, 110B, 110C than the height of the standard anchor seat, selection of an anchor seat 130A, 130B, 130C of an appropriate height can serve to align the spinal rod 120 in the receiving channel and thus minimize, or eliminate, the need to back out the bone anchor 110A, 110B, 110C (and thus avoid potential loss of bone purchase) or over-persuade the rod into the receiving channel (and thus avoid the potential pullout of the bone anchor 110A, 110B, 110C). Proper alignment of the spinal rod 120 within the rod receiving channel through selection of an appropriate height anchor seat 130A, 130B, 130C further reduces “false” locking, which can result in screw toggling or rod slippage.

Due to patient anatomy, typical pedicle screw assemblies having uniform height anchor seats do not permit linear alignment of the rod receiving channels, often in multiple level constructs, and significant spinal rod 120 bending is necessary. The ability to select various height anchor seats 130A, 130B, 130C facilitates increasing or decreasing of lordosis, kyphosis, and coronal plane curves with a straight spinal rod 120, as the curvature of the spinal rod 120 does not need to match the anatomical curve achieved. This can be especially beneficial in Minimally Invasive Spine Surgery and for correcting adult deformities. In another option, the spinal rod 120 is comprised of a curved spinal rod 120, as shown in FIG. 8.

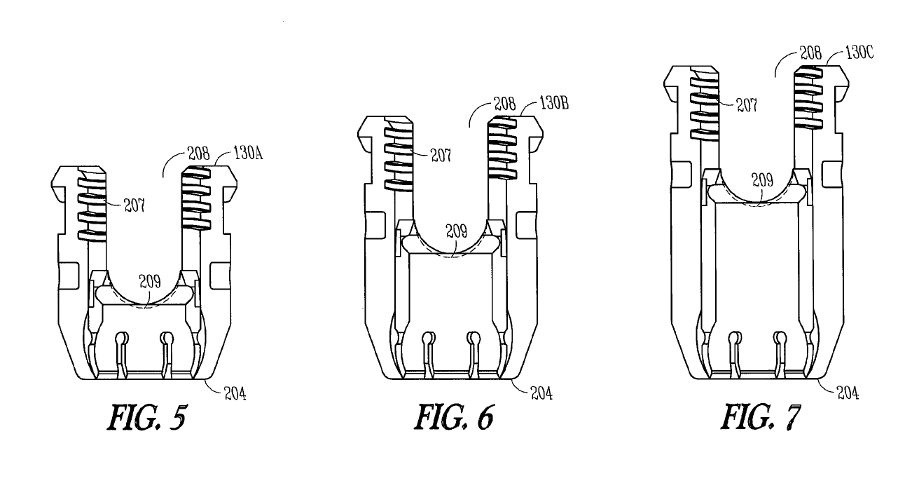

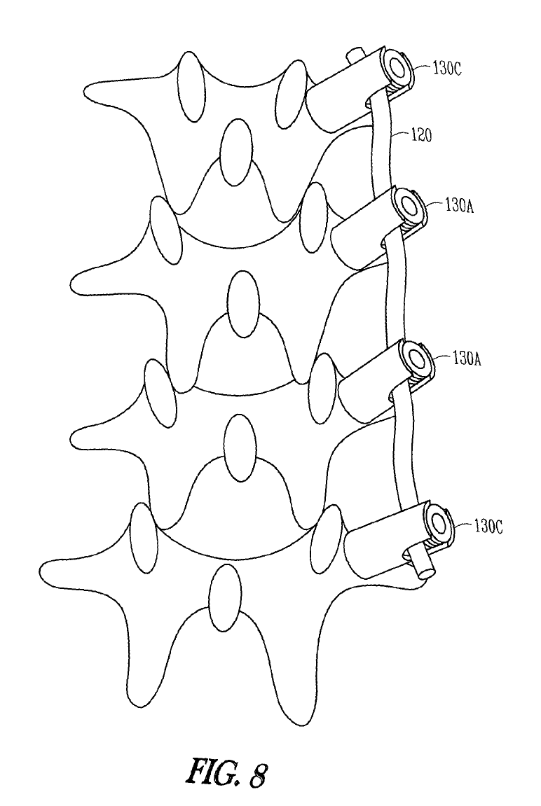

In operation, and in continuing reference to FIGS. 1 and 2, a plurality of bone anchors 110A, 110B, 110C are implanted into the pedicles of a plurality of vertebral bodies. A plurality of anchor seats 110A, 110B, 110C are chosen from a kit that includes at least two which have different heights between the channel distal end 209 of the rod receiving channel and the distal end 204 of the anchor seat 130A, 130B, 130C. For example, the heights can be 5 mm, 9 mm, 13, mm, as shown in FIGS. 5, 6, and 7, respectively. The plurality of anchor seats 130A, 130B, 130C are popped over the heads of the plurality of bone anchors 110A, 110B, 110C. In an option the heads of the bone anchors 110A, 110B, 110C, can be preassembled, and/or modular and provided in a kit. The anchor seats 130A, 130B, 130C are polyaxially angulated with respect to the heads of the bone anchor 110A, 110B, 110C prior to or during the insertion of the spinal rod 120 through the rod receiving channels 208 of the anchor seats 130A, 130B, 130C.

The plurality of locking caps 140A, B, C are coupled to and rotatably advanced within the plurality of anchor seats 130A, 130B, 130C to crush lock the collets 152 around the heads of the bone anchors 110A, 110B, 110C and, thereby, lock the angulation of the anchor seats 130A, 130B, 130C with respect to the bone anchors 110A, 110B, 110C and lock the position of the spinal rod 120 with respect to the plurality of bone anchors 110A, 110B, 110C. The distance between the spinal rod 120 and the heads of the bone anchors 110A, 110B, 110C and, thus, the vertebral bodies to which they are implanted, differs between one or more of the spinal levels due to the surgeon's intraoperative choice of different anchor seats 130A, 130B, 130C having different heights to accommodate optimal offset for a particular spinal level and, thereby, ease the difficulty of complex spinal corrective surgery and achieve full rod reduction and anatomical correction. The amount of the offset can be controlled by the selection of the height of the anchor seats 130A, 130B, 130C. For example, while a standard anchor seat has an approximately 4 mm offset at 25 degrees when used with a 6 mm spinal rod 120 and a 1 mm bone anchor head, an anchor seat with a 4 mm height would have an approximately 5.8 mm offset at 25 degrees (approximately equivalent to a 36 degree offset of the standard anchor seat), and an anchor seat with a 8 mm height would have an approximately 7.5 mm offset at 25 degrees (approximately equivalent to 50 degree offset of the standard anchor seat). In another option, the anchor seat has at least a 1 mm offset. In another option, the offset is in the range of about 4-15 mm.

Further, due to the modularity of the bottom-loading, pop-on anchor seats 130A, 130B, 130C, if the surgeon is unable to completely reduce the spinal rod 120 into a particular anchor seat 130A, 130B, 130C, the surgeon can remove the particular anchor seat 130A, 130B, 130C, such as a first anchor seat assembly, from its bone anchor 110A, 110B, 110C, e.g., using a push-button instrument or a forceps-type grasping instrument, and replace it with an anchor seat 130A, 130B, 130C, such as a second anchor seat, having an appropriate height without necessitating the removal or replacement of its corresponding bone anchor 110A, 110B, 110C. The flexibility to remove and replace any particular anchor seat 130A, 130B, 130C with respect to its corresponding bone anchor 110A, 110B, 110C, without necessitating the removal and replacement of the corresponding bone anchor 110A, 110B, 110C eliminates one of the most time consuming and risky aspects of conventional pedicle screw and rod construct implantation procedures.

The spinal rod 120 can be introduced through the plurality of anchor seats 130A, 130B, 130C through a single small incision due to the flexibility afforded by the different heights and the bottom-loading nature of the polyaxial pedicle screw assemblies.

The various vertical offsets provided to each anchor seat 130A, 130B, 130C of the spine fixation system 100 may increase allowable medial/lateral offset of the spinal rod 120 with respect to a bone anchor 110A, 110B, 110C head. Specifically, the variable height anchor seats 130A, 130B, 130C may permit constructs with less bending of the spinal rod 120 in order to increase or reduce lordosis, kyphosis, and/or coronal plane curves during spinal deformity corrections.

While the present invention has been described with respect to bottom-loading pop-on pedicle screw assemblies, the teachings and the advantages provided by the present invention are applicable to various other pedicle screw systems, including top-loading, preassembled, twist-on, and push-and-turn pedicle screw assemblies. Further, the pedicle screw assemblies of the present invention may include spinal hooks as opposed to bone anchors. Further, the rod receiving channels in the anchor seats, as well as the collets 152 130A, 130B, 130C may be offset with respect to the longitudinal axis of the anchor seat 130A, 130B, 130C.

The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

We claim:

1. A kit comprising:
a plurality of bone anchors, each bone anchor having at least partially polyaxial head and a shaft appending therefrom;
a plurality of anchor seat assemblies, each anchor seat assembly including a proximal end and a distal end, each of the plurality of anchor seat assemblies further including a rod receiving channel having a channel proximal end adjacent the proximal end of the anchor seat assembly and a channel distal end terminating between the anchor seat proximal end and the anchor seat distal end, at least two of the plurality of anchor seat assemblies have different heights between the distal end of the anchor seat assembly and the channel distal end of the rod receiving channel; and
a plurality of locking caps couplable to the plurality of anchor seat assemblies adjacent the anchor seat proximal end adapted to secure a spinal rod with respect to the plurality of bone anchors within one of the rod receiving channels of the plurality of anchor seat assemblies.

2. The kit of claim 1, wherein the anchor seat assembly has an offset of the rod receiving channel of at least 1 mm.

3. The kit of claim 1, wherein the height between the distal end of at least one of the anchor seat assemblies and the channel distal end of the same assembly is 4 mm, and an offset of the rod receiving channel of the same assembly at 25 degrees is approximately 5.8 mm.

4. The kit of claim 1, wherein the height between the distal end of at least one of the anchor seat assemblies and the distal end of the rod receiving channel of the same assembly is 8 mm, and the offset of the rod receiving channel of the same assembly at 25 degrees is approximately 7.5 mm.

5. The kit of claim 1, wherein the anchor seat assemblies are modular, interchangeable seat assemblies.

6. The kit of claim 1, wherein each of the plurality of anchor seat assemblies further including a collapsible collet retained interior to and adjacent the distal end of the anchor seat assembly, the collet adapted to capture and retain the head of one of the plurality of bone anchors

7. A kit comprising:
an elongate spinal rod defined in part by a longitudinal axis;
a plurality of bone anchors, each bone anchor having at least partially polyaxial head and a shaft appending therefrom, the head having a center portion;
a plurality of anchor seat assemblies, each anchor seat assembly including a proximal end and a distal end, each of the plurality of anchor seat assemblies further including a rod receiving channel having a channel proximal end adjacent the proximal end of the anchor seat assembly and a channel distal end terminating between the anchor seat proximal end and the anchor seat distal end;
a plurality of locking caps couplable to the plurality of anchor seat assemblies adjacent the anchor seat proximal end for securing the spinal rod with respect to the plurality of bone anchors within one of the rod receiving channels of the plurality of anchor seat assemblies; and
at least two of the plurality of anchor seat assemblies have different heights between the rod longitudinal axis and the center portion of the head of the bone anchor when the rod is secured within the receiving channel.

8. The kit of claim 7, wherein the height between the rod longitudinal axis and the center portion of the head of the bone anchor when the rod is secured within the receiving channel of the same assembly is 4 mm, and the offset of the rod receiving channel of the same assembly at 25 degrees is approximately 5.8 mm.

9. The kit of claim 7, wherein the height between the rod longitudinal axis and the center portion of the head of the bone anchor when the rod is secured within the receiving channel of the same assembly is 8 mm, and the offset of the rod receiving channel of the same assembly at 25 degrees is approximately 7.5 mm.

10. The kit of claim 7, wherein the bone anchors are pre-assembled with the anchor seat assemblies.

11. The kit of claim 7, wherein the spinal rod is a curved spinal rod.

12. A method comprising:
anchoring a plurality of bone anchors into the plurality of vertebral bodies;
selecting a plurality of anchor seat assemblies, at least two of the plurality of anchor seat assemblies having different heights;
coupling the plurality of anchor seat assemblies to the plurality of bone anchors;
coupling a spinal rod to the plurality of anchor seat assemblies, and
locking the anchor seat assemblies to the spinal rod with a plurality of locking caps.

13. The method of claim 12, wherein coupling at least one of the plurality of anchor seat assemblies to one of the plurality of bone anchors includes popping the bone anchor seat assembly over the bone anchor.

14. The method of claim 12, wherein the bone anchor includes a modular head.

15. The method of claim 12, further comprising anchoring the bone anchors into a lower lumbar portion of a spine, and selecting heights of the anchor seat assemblies so that the spinal rod remains substantially straight subsequent to the locking of the spinal rod to the anchor seat assemblies.

16. The method as recited in claim 12, wherein coupling a spinal rod to the plurality of anchor seat assemblies includes coupling a curved spinal rod to the anchor seat assemblies.

17. The method as recited in claim 12, further comprising at least partially preventing rod bending.

18. The method as recited in claim 12, further comprising at least partially preventing rod reduction.

19. The method as recited in claim 12, further comprising removing one or more first anchor seat assemblies, selecting a second anchor seat assembly having a different height than the removed first anchor seat assembly, and coupling the second anchor seat assembly with the bone anchor.
